(19) United States
(12) Patent Application Publication
(10) Pub. No.: US 2020/0163678 A1
(43) Pub. Date: May 28, 2020

(54) Preloaded Catheter And Clot Retrieval Systems And Methods For Treatment Of Ischemic Stroke

(71) Applicant: MG Stroke Analytics Inc., Calgary (CA)

(72) Inventor: Mayank Goyal, Calgary (CA)

(21) Appl. No.: 16/698,158

(22) Filed: Nov. 27, 2019

Related U.S. Application Data
(60) Provisional application No. 62/771,971, filed on Nov. 27, 2018.

(51) Int. Cl. A61B 17/12; A61M 25/00; A61M 25/01

(52) U.S. Cl. CPC A61B 17/12118; A61M 2025/0293; A61M 25/0125; A61M 25/0067

(57) ABSTRACT

The invention describes systems and methods for retrieving blood clots (thrombi) from patients undergoing endovascular/neurointervention procedures following ischemic stroke. More specifically, a preloaded catheter and clot retrieval system (PCS) effective in positioning a clot retrieval system (CRS) or stent adjacent a clot and ensnaring and removing the clot are described as well as methods of utilizing these devices.

### FIELD OF THE INVENTION

The invention describes systems and methods for retrieving blood clots (thrombi) from patients undergoing endovascular/neurointervention procedures following ischemic stroke. More specifically, a preloaded catheter and clot retrieval system (PCS) effective in positioning a clot retrieval system (CRS) or stent adjacent a clot and ensnaring and removing the clot are described as well as methods of utilizing these devices.

### BACKGROUND OF THE INVENTION

### 1. Background

The human body has an extensive network of blood vessels including both the venous and arterial systems for circulating blood throughout the body. The occurrence and/or development of restrictions to flow within the circulatory system can result in serious medical conditions, the most significant being myocardial infarction and ischemic stroke. The treatment of both conditions (and others involving the circulatory system) continues to evolve with many new techniques and equipment being utilized to effect treatment.

In recent years, a variety of traumatic surgical procedures have been replaced with procedures that involve the use of one or more catheters being advanced through the vascular system of the body to gain access to diagnose and/or treat issues involving the vasculature of a particular organ. For example, ischemic strokes caused by blood clot blockages in the brain, coronary artery blockages within the heart and various heart defects may be treated by advancing catheters to the affected site whence various procedures can be initiated to treat the problem. Stents having various structural and functional properties can be positioned and deployed at a location where intervention is required wherein the specific structure of the stent can allow the treatment of a medical problem. Catheter procedures are also undertaken in other parts of the body including the leg vessels and renal arteries and other complex percutaneous procedures including treatment of valvular heart disease, aortic dissections, dysrhythmias, and management of shunts for dialysis patients can also be performed using catheter systems. Further, complex aneurysms in the brain and other locations are increasingly being treated through a percutaneous endovascular route.

### 2. Stroke Development and Effects

It is known that when a patient experiences a significant ischemic stroke event, those portions of the brain distal to the occlusion that experience a dramatic reduction in blood supply will affect the functioning of large regions of neurons. This reduction in blood supply may cause the patient to become symptomatic, cause the death of regions of the brain and/or put regions of the brain at the risk of dying if not treated quickly. The location and size of the occlusion will result in a wide range of symptoms in the patient and depending on the severity, will ultimately determine how a physician may choose to intervene or not.

Time delays in effecting treatment will typically result in the death of a greater number of neurons. Table 1 shows that in the specific case of acute ischemic stroke, the pace or rate of neural circuitry loss in a typical large vessel supratentorial acute ischemic stroke can be very rapid.

Estimated Pace of Neural Circuitry Loss in Typical Large
Vessel, Supratentorial Acute Ischemic Stroke
Estimated Pace of Neural Circuitry Loss in Typical Large Vessel,
Supratentorial Acute Ischemic Stroke

| | Neurons | Synapses | Myelinated | Accelerated |
|---|---|---|---|---|
| | Lost | Lost | Fibers Lost | Aging |
| Per | 1.2 | 8.3 | 7140 km/4470 | 36 yrs |
| Stroke | billion | trillion | miles | |
| Per Hour | 120 billion | 830 billion | 714 km/447 miles | 3.6 yrs |
| Per Minute | 1.9 million | 14 billion | 12 km/7.5 miles | 3.1 weeks |
| Per | 32,000 | 230 | 200 meters/218 | 8.7 hours |
| Second | | million | yards | |

The numbers represent an average with it also being known that there is a high degree of variability in the above numbers generally depending on the available blood supply to the ischemic region through collateral channels. However, and importantly, delays in making a decision in the order of only a few minutes can have a significant impact on neural circuitry loss and ultimately patient outcome. Further, even slight variations in blood supply can tip the balance and dramatically further increase the rate of cell death if blood supply is reduced or, alternatively prevent neural cell death if blood supply is restored quickly.

2.1. Time to Treatment

The recent paper “Analysis of Workflow and Time to Treatment and the Effects on Outcome in Endovascular Treatment of Acute Ischemic Stroke: Results from the SWIFT PRIME Randomized Controlled Trial” (Radiology, accepted for publication Feb. 24, 2016), and incorporated herein by reference, quantitatively shows that there is a definitive improvement in patient outcome through fast reperfusion. In particular, this study concluded that “aggressive time goals may have contributed to efficient workflow environments”. Further, the study quantifies inter alia that functional independence of a patient was significantly higher when treated quickly (i.e. within 2.5 hours of stroke onset).

Importantly, it is now known that various factors including efficient workflows during a recanalization procedure provided better outcomes.

In diagnosing and treating ischemic stroke, it is important for the physician to know where the vessel occlusion is, how big the occlusion is, where any dead brain tissue (termed “core”) is and the size and location of the brain tissue that may have been affected by the ischemic event but that may potentially be saved (termed “penumbra”).

When responding to acute ischemic stroke, endovascular treatment of acute ischemic stroke due to large vessel occlusion in the anterior circulation is now the standard of care for patients under certain criteria. That is, patients exhibiting particular symptoms (i.e. stroke symptoms of a particular severity) will benefit from early and rapid endovascular intervention to open occluded blood vessels. During various endovascular treatments, a surgeon will advance clot-retrieval (stents) and/or clot-suction devices into the brain's vasculature to the location of the clot where the clot is either withdrawn and/or aspirated from the clot site.

There are many anatomical and situational considerations that can affect the severity and ultimately, treatment of ischemic stroke. Importantly, as described above, while a blood clot may severely affect blood flow to the ischemic area, some blood flow may get to the ischemic area if collateral arteries are functioning to at least partially perfuse the affected area.

The most common large vessel occlusion that is treated by endovascular techniques is the M1 segment of the middle cerebral artery (MCA).

### 3. Recanalization Procedures

Recanalization procedures utilize a wide range of equipment and techniques to access a clot and effect its removal. Generally, the endovascular surgeon will have a number of tools at their disposal including a wide range of guide catheters, microcatheters, microwires, stents and other tools that individually have properties, features and functions that are effective for different procedures and patient presentations.

When an endovascular surgeon deploys a clot retrieval system (CRS) or stent to retrieve a clot, the stent is generally conveyed to the clot within a microcatheter in a compressed state. The typical modern stent is a fine mesh of wires that, once expanded, form a small network of crisscrossing wires that upon deployment penetrate/ensnare the surface of the clot and otherwise engage with the clot to allow the clot to be drawn proximally from the occlusion site and removed from the body. Generally, engagement of the wires with the clot requires that the wires penetrate the surface of the clot in a manner where sufficient friction and/or interfacial forces between the clot and wires exist to wholly and fully allow the clot to be withdrawn and aspirated from the body. Generally, the mesh of wires can be open or closed cell designs where most closed cell design stents will foreshorten as they are deployed.

3.1. Recanalization Procedures

Recanalization procedures are most commonly performed by gaining access to the arterial vascular system through the patient's groin area by puncturing the common femoral artery. An arterial sheath is inserted.

Then, under fluoroscopic (x-ray) guidance, a catheter system (usually a co-axial system including a guide catheter or balloon guide catheter and diagnostic catheter) is advanced through the descending aorta to reach the aortic arch.

The diagnostic catheter is shaped and is used to hook the vessel of interest and, with the help of a guidewire, the diagnostic catheter is advanced to the relevant carotid artery. Subsequently, the guide catheter/balloon guide catheter is advanced over the diagnostic catheter such that the tip is in the relevant internal carotid artery.

At this stage, the diagnostic catheter and wire are removed.

Subsequently, catheters that are designed for intracranial access are advanced through the guide catheter. This will typically consist of one of two approaches:
a. a microcatheter and a microwire; or,
b. a tri-axial system comprising of a distal access catheter (DAC), a microcatheter and a microwire.

For approach a: once the clot has been crossed by the microcatheter and microwire, the microwire is removed and a stent is carefully and slowly deployed across the clot. While aspirating through the guide catheter (with the balloon inflated if using a balloon guide catheter (BGC)), the stent is withdrawn to capture the clot and establish reperfusion.

For approach b: the DAC is placed proximal to the clot. In approach b1: the microcatheter is used to cross the clot and, after removal of the microwire, a stent is deployed. Then the stent and DAC are typically withdrawn together, while aspirating from the DAC. In approach b2: a stent is not used and an attempt is made to directly capture the clot by aspirating through the DAC.

Approach a and b. may be combined together where a DAC is introduced through the BGC and then the microcatheter and stent through the DAC. In this approach, double suction is applied: through the DAC and through the BGC.

All of these approaches typically require accessing the carotid artery through the aortic arch.

As noted above, it is known that stroke typically affects the elderly and with increasing age, there is an increase in tortuosity of various vessels including the aortic arch and/or ophthalmic artery (OA), making it tough to move catheter systems through these areas. In particular, a highly tortuous OA can be difficult to advance catheter systems through as high-bend angles and friction may cause distal tips of coaxial catheters to partially separate and create additional friction.

As described in the inventor's co-pending application U.S. Ser. No. 14/809,867 and incorporated herein by reference, catheter systems having tapered sections may be effectively used to improve movement through these tortuous arterial systems.

### 4. Catheter Design and Performance

As mentioned above, there are generally two classes of catheters used in cerebral procedures, namely diagnostic and guide catheters. Diagnostic catheters are generally those used to gain access to an area of interest whereas guiding catheters are used to support and guide additional equipment including diagnostic catheters, guidewires, balloons, other catheters etc. as may be required for a particular surgical technique.

Typical diagnostic catheters will range from 4 F to 6 F (French) and have lengths of 65-125 cm. They may have braided wall structures and they will generally have a soft tip with a range of shapes formed into the tip.

Guide catheters are generally larger (e.g. 6-8 F) and are 80-100 cm in length. They generally have reinforced construction with a significantly stiffer shaft to provide back-up (i.e. retro) support for the advancement of any additional equipment as listed above.

From an anatomical perspective, catheters generally pass through different zones of the vasculature, namely the abdominal and thoracic vasculature between the femoral artery and aortic arch (approximately 50-75 cm), the cervical vasculature (approximately 15-20 cm) and the cephalic/cerebral vasculature (approximately 10-15 cm). The vessels progressively narrow from 2 cm in the aorta down to 3 mm and smaller in the cerebral vessels.

4.1. Catheter Construction

The choice of a particular catheter or system of catheters may be determined by the skill and experience of a particular interventionist.

Some typical properties of different catheters are summarized in Table 2.

TABLE 2
Summary of Catheter Properties

| | | Diameter and | Typical Tip |
|---|---|---|---|
| Catheter | Body Properties | Typical Length | Features |
| Guide | Usually quite stiff | 6-8F | May have |
| Catheter | Atraumatic tip | Extracorporeal + | balloon |
| | Supports and guides | Groin to Carotid | |
| | other catheters | 80-100 cm | |
| | Double lumen if Balloon | | |
| | Guide Catheter (BGC) | | |
| Diagnostic | Variable Tip Stiffness | 4-6F | Soft Tip |
| Catheter | Variable Tip Shapes | Extracorporeal + | Multiple |
| | Torquable | Groin to Carotid | Shapes |
| | | 100-125 cm | |
| Microcatheter | Soft Tip | 1-5-2.5F | Rounded |
| | Pushable | Goes through | Soft Tip |
| | Trackable | the guide | |
| | | catheter | |
| | | Travel to | |
| | | intracranial | |
| | | vessels (over a | |
| | | microwire) and | |
| | | to beyond the | |
| | | clot. | |
| | | 150 cm | |
| Guide Wire | Pushable | 1F | Rounded |
| | Torquable | Travels inside of | |
| | | diagnostic | |
| | | catheter or guide | |
| | | catheter (used to | |
| | | advance these | |
| | | catheters to the | |
| | | cervical carotid | |
| | | artery) | |
| | | 150-300 cm | |
| Reperfusion | Multizone (may be up to | 4-6F (diameter | Rounded |
| Catheter | 12-15 zones) | may be more | Soft Tip |
| | Increasing level of | proximally to | Challenging |
| | softness distally to allow | allow for better | design to |
| | the catheter to negotiate | suction. | prevent |
| | significant tortuosity and | | ovalization |
| | remain atraumatic | | during passing |
| | Distal transition zones | | through |
| | may extend for 30- | | significant |
| | 40 cm) | | curvature and |
| | Enables two-way Fluid | | while |
| | Flow | | applying |
| | Pushable | | suction |
| Stent | Integrated Clot Retrieval | Very small in its | Integrated |
| | System | collapsed state | Clot |
| | Pushable | (travels through | Retrieval |
| | | microcatheter). | System |
| | | In expanded | |
| | | state: 3-6 mm | |
| | | Extracorporeal + | |
| | | Groin to | |
| | | Occlusion | |
| | | 180 cm | |
| Microwire | Pushable | 0.25-0.4 mm | Round soft tip |
| | Torquable | 180-200 cm | |
| | Soft, atraumatic tip | travels through | |
| | | microcatheter | |
| | | extracorporeal to | |
| | | intracranially | |
| | | (beyond the clot) | |

While the above procedures are effective, there continues to be a need for catheter systems that make it easier to move through tortuous vessels in the brain while simultaneously reducing the number of steps in certain procedures such that the procedure can be completed in a shorter time period. In particular, there has been a need for improved catheter systems that have a wider range of physical properties that reduce the need to withdraw catheters from the body and insert other catheters. More specifically, there has been a need for catheter systems that do not require additional steps of withdrawing a microwire fully and then a second step of pushing a stent to an occlusion step.

### SUMMARY OF THE INVENTION

In accordance with the invention, there are provided systems and methods for improving the efficiency of surgical procedures using catheter systems to move from an entry point to a location in the body where a treatment or diagnostic procedure may be completed.

In a first aspect, the invention provides a preloaded catheter system (PCS) for carrying a stent within a vasculature to a deployment site comprising: an outer catheter system (OCS) having a distal tip section having a distal lumen diameter for operative containment of a microwire; a stent holding section proximal to the distal tip section, the stent holding section having a lumen volume and diameter for reversibly retaining a compressible wire stent and enabling operative use of a microwire through the stent holding section; a proximal section having a proximal lumen diameter for operative containment of a microwire; a stent operatively retained within the stent holding section; where the microwire includes a microwire lock adjacent a distal tip of the microwire and where the microwire lock is operatively engageable with a stent lock adjacent a proximal end of the stent; and where the microwire lock and the stent lock are engageable through application of a proximal pressure applied to the microwire and where, once engaged, application of a distal pressure enables deployment of the stent from the distal tip of the OCS.

In one embodiment, the OCS includes a distal outer surface taper between the distal tip section and stent holding section.

In another embodiment, the OCS includes a proximal outer surface taper between the proximal section and the stent holding section.

In one embodiment, the distal outer tapered surface has dimensions to facilitate advancement of the OCS and a distal access catheter (DAC) through a tortuous section of a human vasculature.

In another embodiment, the proximal outer tapered surface has outer dimensions to facilitate advancement of a distal access catheter (DAC) through a tortuous section of a human vasculature without separation of a distal tip of the DAC from an outer surface of the stent holding section.

In yet another embodiment, the stent is a clot retrieval stent.

In one embodiment, the stent includes a plurality of wire frame openings defining separate zones and where each zone has wire frame openings of a different average diameter.

In another aspect, the invention provides a stent for operative deployment from a preloaded catheter system as described above including: a compressible wire body expandable within the stent holding section, the wire body defining a plurality of wire openings; a proximal stent lock engageable with a corresponding microwire lock; where prior to engagement, the proximal stent lock enables operative movement of a microwire within the stent holding section; and, where after engagement of the stent lock with microwire lock, the stent is deployable in distal direction from the stent holding section.

In one embodiment, the stent lock includes a hollow ring enabling operative containment of a microwire within the hollow ring and where the hollow ring includes at least one surface for locking engagement with the microwire lock.

In another aspect, the invention provides a microwire for operative use within an outer catheter system (OCS) as described herein comprising: a microwire body having a distal tip; and, a microwire lock adjacent the distal tip for engagement with a stent lock; wherein the microwire lock has dimensions for operative movement within the OCS.

In another aspect, the invention provides a lock system for locking a microwire to a stent comprising: a microwire lock operatively connected to a microwire adjacent a distal tip of the microwire, the microwire lock having at least one microwire locking surface; a stent lock operatively connected to a proximal end of an expandable stent, the stent lock including a ring surrounding a microwire and having at least one stent locking surface for engagement with the at least one microwire locking surface; and where the microwire lock and stent lock are engageable by application of a proximal pressure to the microwire lock to draw the at least one microwire locking surface into the ring to engage the at least one microwire locking surface with the at least one stent locking surface.

In a further aspect, the invention provides a method of conveying a stent through a vasculature from an entry point to a deployment site utilizing a preloaded catheter system (PCS) having: a distal tip section having a distal lumen diameter for operative containment of a microwire; a stent holding section proximal to the distal tip section, the stent holding section having a lumen volume and diameter for reversibly retaining a compressible wire stent and enabling operative use of a microwire through the stent holding section; a proximal section having a proximal lumen diameter for operative containment of a microwire; a stent operatively retained within the stent holding section; where the microwire includes a microwire lock adjacent a distal tip of the microwire and where the microwire lock is operatively engageable with a stent lock adjacent a proximal end of the stent and where the microwire lock and the stent lock are engageable through application of a proximal pressure applied to the microwire and where, once engaged, application of a distal pressure enables deployment of the stent from the distal tip of the OCS, the method comprising the steps of: a) introducing the PCS into the vasculature; b) successively advancing the microwire and OCS through the vasculature to a deployment site; c) partially withdrawing the microwire (MW) proximally to engage the MW lock with the stent lock; and, d) deploying the stent by withdrawing the OCS relative to the stent to effect deployment of the stent from the distal tip.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention; however, the scale of the drawings may be relied upon for supporting the relative position of described components with respect to one another. Similar reference numerals indicate similar components.

FIG. 1A is a schematic sketch of a portion of brain vascular anatomy showing the ophthalmic artery (OA), intracranial internal carotid artery (IICA), anterior cerebral artery (ACA), M1 segment of the middle cerebral artery and M2 segment of the middle cerebral artery.

FIGS. 1B and 1C are schematic sketches as in FIG. 1A showing a stent device entangled with and withdrawing a clot; FIG. 1C shows a common issue of a stent dropping a clot when negotiating a tortuous region of the cerebral anatomy.

FIG. 2 is a schematic diagram of a catheter system in accordance with the prior art where the catheter system has an expanded tapered section adjacent the distal tip to assist in negotiating tortuous regions of the cerebral anatomy.

FIG. 3 is a schematic diagram in accordance with the invention showing a preloaded catheter system (PRS).

FIG. 3A is a schematic diagram in accordance with the invention showing a preloaded catheter system with the stent lock engaged.

FIG. 3B is a schematic diagram in accordance with the invention showing a preloaded catheter system with the stent lock engaged and partially emerged from the distal tip of the PRS.

FIG. 3C is a schematic diagram in accordance with the invention showing a preloaded catheter system with the stent lock engaged and emerged from the distal tip of the PRS.

### DETAILED DESCRIPTION OF THE INVENTION

### 5. Introduction

With reference to the figures, systems and methods for retrieving blood clots via endovascular intervention are described. More specifically, systems adapted for advancing and deploying stents within a patient's vasculature following ischemic stroke are described.

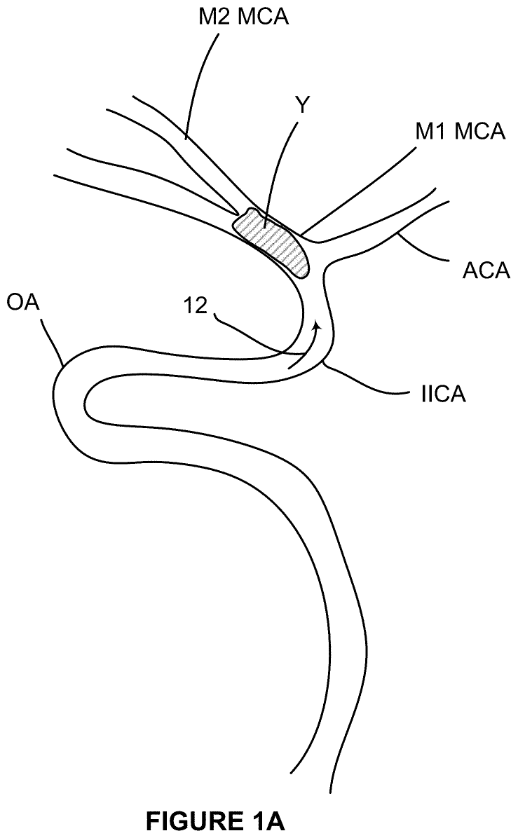

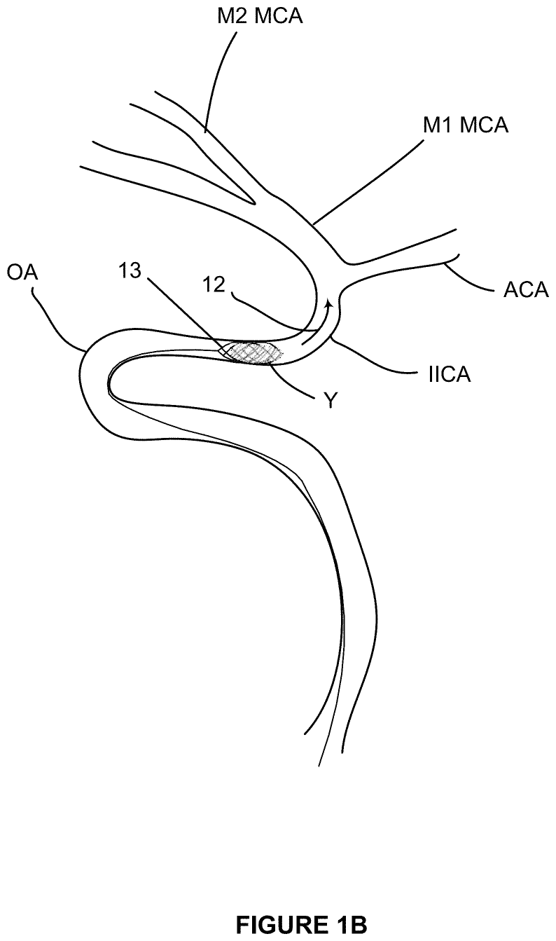

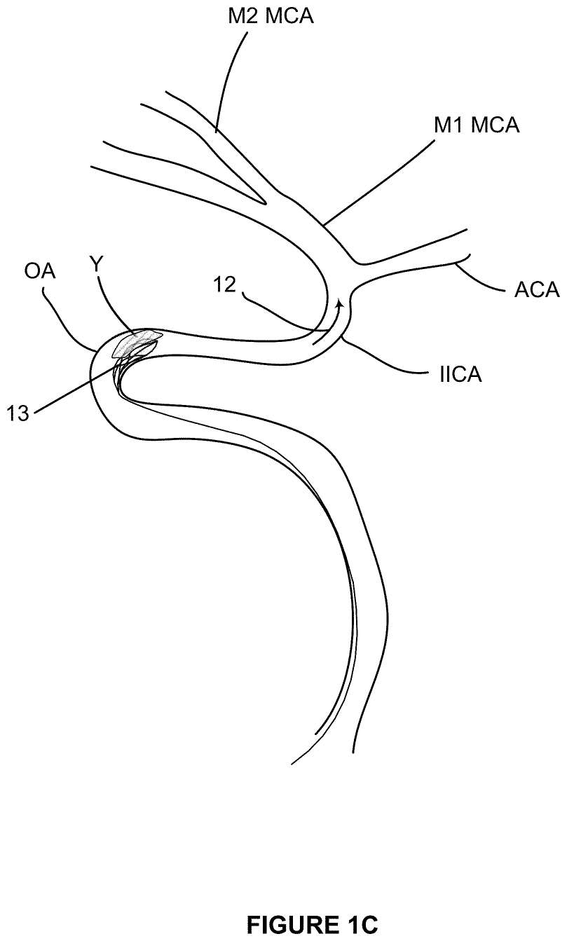

FIG. 1A is a schematic diagram of brain vascular anatomy showing the intracranial internal carotid artery (IICA), anterior cerebral artery (ACA), M1 segment of the middle cerebral artery and M2 segment of the middle cerebral artery. A clot Y is shown within the M1 MCA with arrow 12 showing the direction of blood flow prior to any procedure. FIG. 1A also shows a tortuous region (e.g. the ophthalmic artery (OA)) which is a region that can be difficult both to advance and withdraw catheter systems through.

FIG. 1B illustrates a simplified example of a surgeon withdrawing a stent 13 that has entangled clot Y. For the purposes of illustration, the stent 13 may be flattened as it is drawn through a tortuous section resulting in the partial or complete release of the clot Y (FIG. 10) as the wires of the stent move with respect to one another thus changing/enlarging the stent openings.

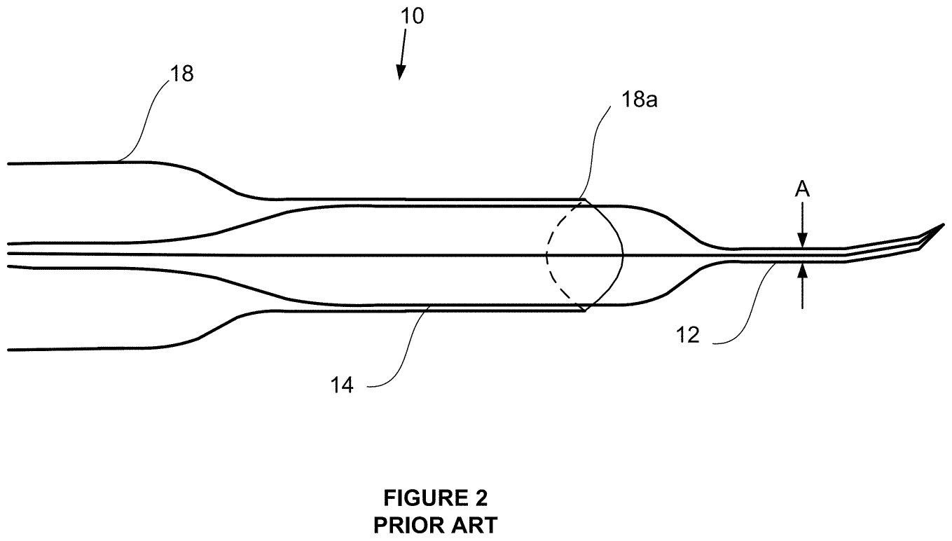

FIG. 2 shows a catheter system in accordance with the prior art and described in US patent application Ser. No. 14/809,867, incorporated herein by reference. As shown, this catheter system 10 includes a distal tip section 12, an expanded section 14 and a proximal support section 16. The catheter system may include a distal access catheter 18 where a middle portion 14b has a diameter that fits into and supports a distal end 18a of the DAC.

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

### 6. Preloaded Catheter System Overview

The invention provides a catheter system that enables problems identified above to be overcome. In particular, the invention describes a system/assembly of a preloaded stent, microwire, and catheter system, referred to herein as a preloaded catheter system (PCS) 20 that can be effective in reducing the number of steps and hence time required to effect stent deployment, clot capture and recovery.

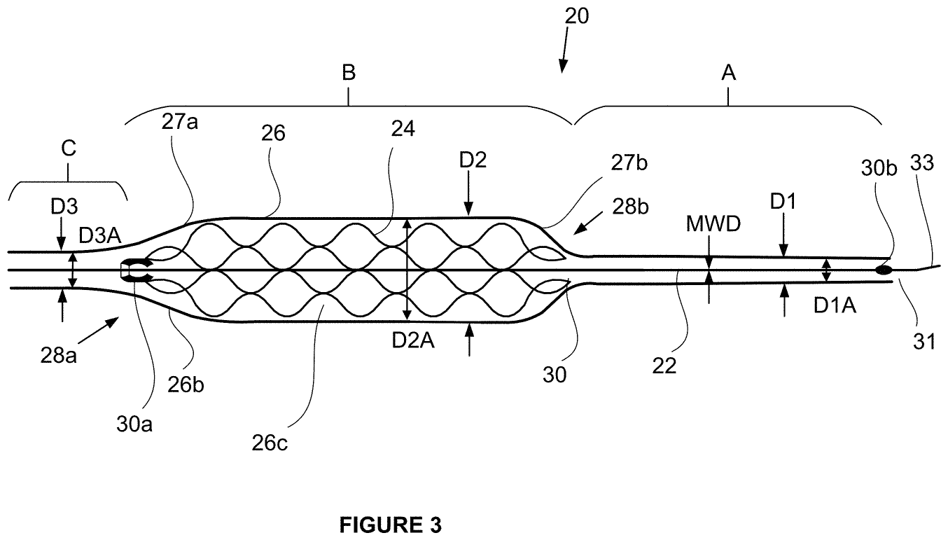

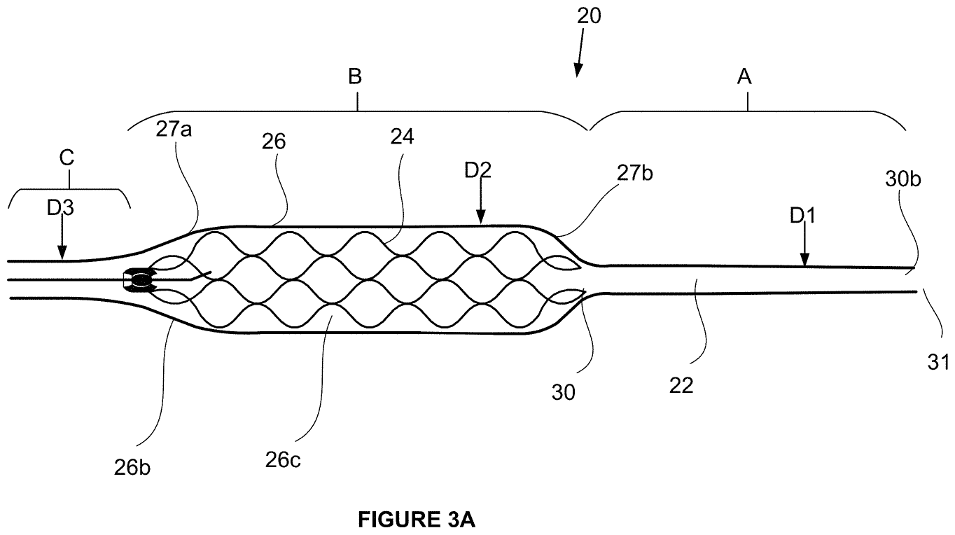

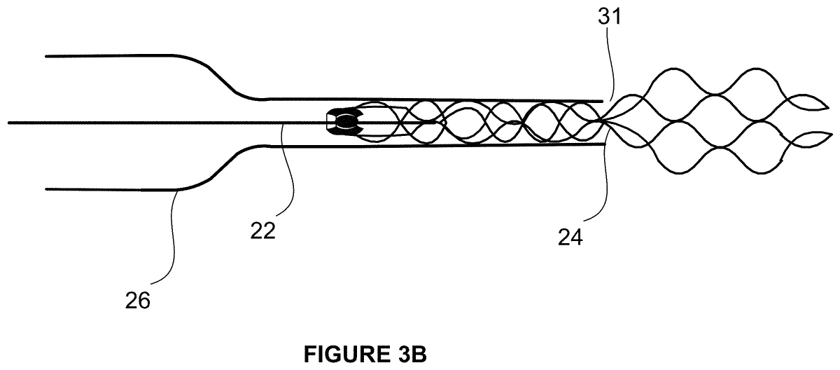

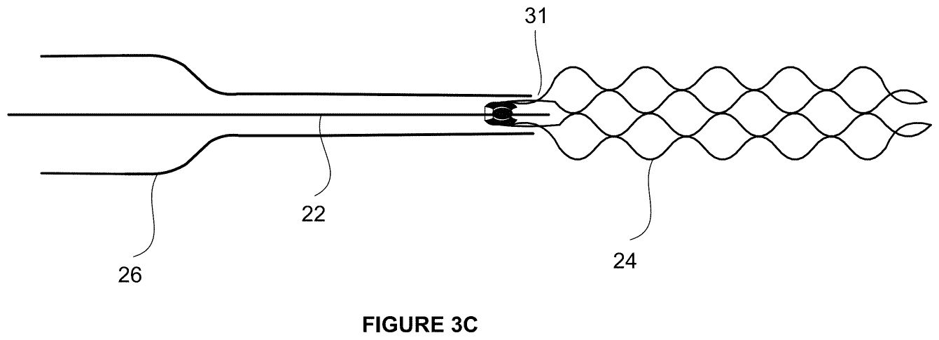

As shown in FIGS. 3, 3A, 3B and 3C, the PCS 20 includes a microwire (MW) 22, a stent 24 and an outer catheter system (OCS) 26. Generally, during a surgical procedure and after the surgeon has gained access to the appropriate carotid artery, the PCS is advanced towards a clot, via sequential and iterative movement of the MW and OCS. That is, the surgeon will sequentially steer and advance the MW towards the clot and follow the MW with the OCS.

When the clot is reached, the MW is positioned distal to the clot and the distal tip of the OCS also advanced past the clot. As will be explained in greater detail below, the MW is partially withdrawn to lock and engage with the stent, which is retained within the OCS. After the MW locks and engages the stent (FIG. 3A) by applying a distal pressure to the MW, the OCS is withdrawn such that the stent is deployed from the distal tip of the OCS (FIG. 3B).

After the stent has been deployed, the surgeon waits a period of time to allow the clot to entangle with the stent. When the clot has become entangled with the stent, both the OCS and stent carrying the clot will be withdrawn proximally into an aspiration catheter to substantially complete the procedure.

Further description of each component follows:

### 7. Outer Catheter System (OCS) 26

As shown in FIG. 3 (not to scale), the OCS is generally comprised of 3 zones including a distal tip zone (A), a stent preload zone (B) and a proximal zone (C).

7.1. Distal Tip Zone (A)

The distal tip zone generally functions as a microcatheter, that is zone A is characterized as having an outer diameter of 1.5-2.5 F and inner dimensions enabling passage of a MW 22 and compressed stent through this zone. Zones A and C will typically have an inner diameter D1A, D3A of about 0.6 mm. The MW will typically have an outer diameter (MWD) of about 0.35 mm.

Zone A will be typically be 6-8 cm in length from the distal tip 31.

7.2. Stent Preload Zone (B)

The stent preload zone (B) generally functions to provide a) tapered outer surfaces 27a, 27b to facilitate movement of the OCS through tortuous regions and b) an inner stent cavity 26c to hold a stent 24 during distal movement of the PCS.

The distal taper zone 27b tapers from the zone A diameter D1 (1.2-2.5 F) to the zone B diameter D2 (typically 6-8 F). Zone B is preloaded with a stent 24 within stent chamber 26c that operatively retains the stent in a partially expanded state. The proximal side of zone B tapers from D2 to D3 in zone C. The inner diameter D2A of the lumen of zone B is in the range of 1 mm.

The typical length of a stent is about 2-4.5 cm and will have a OD of about 4-6 mm when fully expanded; hence, the stent chamber will have a substantially corresponding length and a lumen diameter large enough to accommodate both the MW and partially collapsed stent; thus, will be in the range of 1 mm or roughly about 25% of the expanded diameter of the stent. Importantly, the distal and proximal ends of the stent chamber will have tapered surfaces to enable the stent to smoothly expand and compress as the stent is placed within or transitioned out of the stent chamber.

Once placed within the stent chamber, the stent will thus expand radially against the inner walls of the stent chamber thus maintaining a void volume within the interior of the stent chamber.

7.3. Stent and Microwire

A stent 24 is configured for functional placement within the stent chamber. Initially, that is at the beginning of a procedure, the stent is not engaged or locked with the MW. Immediately prior to deployment, the stent is connected to the MW (explained below) and can thereafter be a) collapsed and deployed from the OCS through zone A and b), if necessary withdrawn back into the OCS through zone A.

The MW is substantially a typical MW having the length and properties to function as a MW within a microcatheter including a tip 33 that may be of a fixed shape or formable by the surgeon. Other properties of torqueability and steerability will be present in the MW without interfering with the stent in zone B. The MW will include a MW lock 30b configured to the MW approximately 7-10 mm from the distal tip of the MW. The MW lock will not interfere with the normal operation of the MW within zone A.

In order to enable the catheter positioning procedure and the lock connection, during advancement of the PCS the MW and OCS can be moved with respect to one another where the stent remains statically “fixed” within the stent chamber from radial pressure exerted by its wire frame against the inner walls of the lumen of zone B.

At the time of stent deployment, the surgeon draws back on the MW to engage lock components 30a, 30b on the stent and MW.

As shown, the stent has a proximal end 28a and a distal end 28b. In order to enable proximal movement of a deployed stent and to pull an engaged clot proximally, the proximal end of the stent must engage with the MW lock. Accordingly, in one embodiment, the MW includes a stent lock 30a and a MW lock 30b that can engage together to lock the two pieces together. The MW lock 30b may be an expanded volume on the MW that engages with a corresponding and mating stent lock 30a on the proximal end of the stent.

In operation, by gently pulling the MW proximally, the MW lock 30b passes through the interior of the stent and is guided into the stent lock 30a. As the two pieces engage, proximal pressure on the stent lock will draw the stent proximally and against the inside proximal taper 27a of the OCS. Various resistive forces can be utilized to effect permanent engagement of the MW and stent lock together.

Once engaged, the MW can be held and the OCS can then be pulled proximally such that the stent will collapse “forwardly” into zone A. As proximal pressure on the OCS is maintained, the stent will fully compress within zone A, progress through zone A and emerge from the distal tip 31 as shown in FIGS. 3B and 3C.

If clot capture is successful, the PCS, deployed stent and clot are withdrawn to an aspiration catheter (not shown).

If clot capture is not successful, and/or it becomes necessary to withdraw the stent back into the OCS, proximal pressure on the MW will pull and collapse the stent back into zone A of the OCS.

The locking system may be implemented in a number of ways.

It is important that the stent does not separate from the MW after deployment. Hence, the locking system should provide greater security against failure of the lock in the proximal direction rather than the distal direction.

Suitable locking systems may include various combinations of forces to ensure connection and may include devices incorporating systems having positive displacement detents, latches, twist-locks and others. These can include proximal pressure and torque via the MW as well as distal pressure provided by the OCS proximal tapered surface (including potentially additional interior surfaces) and/or an external pressure provided by a tertiary catheter external to the proximal tapered surface that could be used to effect a squeezing pressure against the proximal tapered surface to narrow the proximal tapered surface and thus provide a greater resistive force to the locking system.

The stent may have a plurality of zones as described in Applicant's co-pending application 62/696,641, incorporated herein by reference.

Further still, the OCS may enable the use of different stents having properties that would otherwise be limited by introducing a sheathed stent and having to push that stent the full distance from the extracorporeal access point to the clot site.

### 8. Factory Assembly

As described, the OCS includes a stent chamber (zone B) having an outer diameter D2 and adjacent zones A and C, having outer diameters D1 and D3.

Generally, the PCS is assembled in accordance with the following general steps:
a. A loading MW (not shown) is threaded through a stent. The loading MW has a non-locking surface that can apply a proximal force against the distal side of the stent lock.
b. The loading MW with threaded stent is fed into the distal end of an empty OCS and fully pushed to the proximal end of the OCS.
c. The loading MW is continued to be pulled proximally to gently draw the stent into the OCS through the distal tip 31 and into the stent chamber.
d. When correctly seated, the loading MW is fully withdrawn from the OCS through the distal tip.
e. The MW 22 (having a MW lock) is loaded into the distal tip and pushed fully through to proximal end of the OCS. Markers on the proximal and/or distal end of the OCS and MW will ensure the correct linear position of the MW lock.
f. Additional catheters (guide, balloon guide, aspiration) may be loaded from the proximal end of the PCS in some embodiments or packaged alongside the PCS.

It is understood that the system may be manufactured with a number of sizes and/or catheter performance features as understood by those skilled in the art.

In another aspect, the PCS may be used for the placement of stents used in the treatment of aneurysms, as described in Applicant's co-pending U.S. provisional application 62/616,980, incorporated herein by reference. In these procedures, the OCS would be adapted to carry an aneurysm stent. In this case, as aneurysm stents are left in place, a MW would be used to push the stent from zones B and A of the OCS. Accordingly, in this case, a push surface would be positioned proximal to the proximal end of the stent. In this case, after positioning the tip of the OCS at the location of deployment, the original MW (i.e. a standard MVV) may be withdrawn and a separate pushing MW may be introduced. Importantly, it should be noted that as aneurysm treatment procedures are not emergency procedures, the extra step of withdrawing a MW and introducing a pushing MW is not significant.

Although the present invention has been described and illustrated with respect to preferred embodiments and preferred uses thereof, it is not to be so limited since modifications and changes can be made therein which are within the full, intended scope of the invention as understood by those skilled in the art.

1. A preloaded catheter system (PCS) for carrying a stent within a vasculature to a deployment site comprising:
an outer catheter system (OCS) having
a distal tip section having a distal lumen diameter for operative containment of a microwire;
a stent holding section proximal to the distal tip section, the stent holding section having a lumen volume and diameter for reversibly retaining a compressible wire stent and enabling operative use of a microwire through the stent holding section;
a proximal section having a proximal lumen diameter for operative containment of a microwire;
a stent operatively retained within the stent holding section;
where the microwire includes a microwire lock adjacent a distal tip of the microwire and where the microwire lock is operatively engageable with a stent lock adjacent a proximal end of the stent;
and where the microwire lock and the stent lock are engageable through application of a proximal pressure applied to the microwire and where, once engaged, application of a distal pressure enables deployment of the stent from the distal tip of the OCS.

2. The system as in claim 1 where the OCS includes a distal outer surface taper between the distal tip section and stent holding section.

3. The system as in claim 1 where the OCS includes a proximal outer surface taper between the proximal section and the stent holding section.

4. The system as in claim 2 where the distal outer tapered surface has dimensions to facilitate advancement of the OCS and a distal access catheter (DAC) through a tortuous section of a human vasculature.

5. The system as in claim 2 where the proximal outer tapered surface has outer dimensions to facilitate advancement of a distal access catheter (DAC) through a tortuous section of a human vasculature without separation of a distal tip of the DAC from an outer surface of the stent holding section.

6. The system as in claim 1 where the stent is a clot retrieval stent.

7. The system as in claim 6 where the stent includes a plurality of wire frame openings defining separate zones and where each zone has wire frame openings of a different average diameter.

8. A stent for operative deployment from a preloaded catheter system as in claim 1 comprising:
a compressible wire body expandable within the stent holding section, the wire body defining a plurality of wire openings;
a proximal stent lock engageable with a corresponding microwire lock;
where prior to engagement, the proximal stent lock enables operative movement of a microwire within the stent holding section; and,
where after engagement of the stent lock with microwire lock, the stent is deployable in distal direction from the stent holding section.

9. The stent as in claim 8 where the stent lock includes a hollow ring enabling operative containment of a microwire within the hollow ring and where the hollow ring includes at least one surface for locking engagement with the microwire lock.

10. A microwire for operative use within an outer catheter system (OCS) as described in claim 1 comprising:
a microwire body having a distal tip;
a microwire lock adjacent the distal tip for engagement with a stent lock;
wherein the microwire lock has dimensions for operative movement within the OCS.

11. A lock system for locking a microwire to a stent comprising:
a microwire lock operatively connected to a microwire adjacent a distal tip of the microwire, the microwire lock having at least one microwire locking surface;
a stent lock operatively connected to a proximal end of an expandable stent, the stent lock including a ring surrounding a microwire and having at least one stent locking surface for engagement with the at least one microwire locking surface;
and where the microwire lock and stent lock are engageable by application of a proximal pressure to the microwire lock to draw the at least one microwire locking surface into the ring to engage the at least one microwire locking surface with the at least one stent locking surface.

12. A method of conveying a stent through a vasculature from an entry point to a deployment site utilizing a preloaded catheter system (PCS) having:
a distal tip section having a distal lumen diameter for operative containment of a microwire;
a stent holding section proximal to the distal tip section, the stent holding section having a lumen volume and diameter for reversibly retaining a compressible wire stent and enabling operative use of a microwire through the stent holding section;
a proximal section having a proximal lumen diameter for operative containment of a microwire;
a stent operatively retained within the stent holding section;
where the microwire includes a microwire lock adjacent a distal tip of the microwire and where the microwire lock is operatively engageable with a stent lock adjacent a proximal end of the stent and where the microwire lock and the stent lock are engageable through application of a proximal pressure applied to the microwire and where, once engaged, application of a distal pressure enables deployment of the stent from the distal tip of the OCS, the method comprising the steps of:
a. introducing the PCS into the vasculature;
b. successively advancing the microwire and OCS through the vasculature to a deployment site;
c. partially withdrawing the MW proximally to engage the MW lock with the stent lock; and,
d. deploying the stent by withdrawing the OCS relative to the stent to effect deployment of the stent from the distal tip.
